(19) United States
(12) Patent Application Publication
(10) Pub. No.: US 2011/0262491 A1
(43) Pub. Date: Oct. 27, 2011

(54) EMULSIONS AND METHODS OF MAKING NANOCARRIERS

(75) Inventors: Mark J. Keegan, Groton, MA (US); Christopher E. Mooney, Norfolk, MA (US)

(73) Assignee: Selecta Biosciences, Inc., Watertown, MA (US)

(21) Appl. No.: 13/084,662

(22) Filed: Apr. 12, 2011

Related U.S. Application Data
(60) Provisional application No. 61/323,141, filed on Apr. 12, 2010.

(51) Int. Cl. A61K 9/14; A61K 38/02; A61K 39/39; A61P 37/04; A61K 31/7088; A61K 39/00; B82Y 5/00

(52) U.S. Cl. 424/400; 514/44 R; 514/1.1; 424/184.1; 424/278.1; 977/773; 977/906

(57) ABSTRACT

This invention relates, in part, to methods of using emulsions for making synthetic nanocarriers and the synthetic nanocarriers formed by such methods.

### RELATED APPLICATIONS

This application claims the benefit under 35 U.S.C. §119 of U.S. provisional application 61/323,141, filed Apr. 12, 2010, the entire contents of which are incorporated herein by reference.

### FIELD OF INVENTION

This invention relates to methods of using emulsions for making synthetic nanocarriers and the synthetic nanocarriers formed by such methods.

### BACKGROUND OF INVENTION

An emulsion is a mixture of two or more immiscible fluids, with tiny particles or “droplets” of one liquid suspended in another. Chemically, they are colloids where both phases are fluids, e.g., liquids. In an emulsion, one liquid (the dispersed phase) is dispersed in the other (the continuous phase). The boundary between these phases is called the interface.

Emulsions are unstable and thus do not form spontaneously. Energy input through shaking, stirring, high shear mixing, rotating membrane, homogenizing, or other such processes are needed to initially form an emulsion. Over time, emulsions tend to revert to their separate stable phases. A classic example of an emulsion is oil and water when mixed under vigorous agitation. However, when the agitation is stopped, the two liquids separate and the emulsion breaks down.

An emulsifier is a substance which stabilizes an emulsion. Stabilized emulsions do not separate out or separate out more slowly after a change in conditions like temperature or over time. One class of emulsifiers is known as surface active substances, or surfactants.

Whether an emulsion turns into a water-in-oil emulsion or an oil-in-water emulsion depends on the volume fraction of both phases and on the type of emulsifier. Generally, emulsifiers tend to promote dispersion of the phase in which they do not dissolve very well.

A double emulsion can be thought of as an emulsion within an emulsion. To make a double emulsion, a first emulsion may be formed from a first fluid and a second fluid that are substantially immiscible. The first emulsion comprises the first fluid present as individual, dispersed droplets or within a continuous phase which is the second fluid. Next, the first emulsion is combined with a third fluid which is substantially immiscible with the continuous phase second fluid. The emulsion formed from this combination is individual dispersed droplets of the second fluid contained within the third fluid. These droplets of the second fluid themselves contain droplets of the first fluid. Thus, in a double emulsion, a third fluid continuous phase contains “parent” droplets of a second fluid, each of which in turn contains “child” droplets of a first fluid. This nesting process may be repeated even more times (e.g., with a fourth fluid, a fifth fluid, etc.), creating triple emulsions, quadruple emulsions, etc.

Emulsions are important in many applications, such as food, beverage, health and beauty aids, paints and coatings, pharmaceuticals, etc. The present invention involves the use of such emulsions to form synthetic nanocarriers.

### SUMMARY OF INVENTION

The inventors discovered unexpectedly that molecules can interact undesirably with one another during a synthetic nanocarrier forming process. The molecules can interact with one another causing one or both to precipitate prematurely out of solution, negatively affecting the desired synthetic nanocarrier. Similarly, a first molecule once dissolved in solution can affect the solubility of a second molecule when introduced into that solution, negatively affecting the concentration of the second molecule in the desired synthetic nanocarrier. In one instance, the inventors discovered surprisingly that reducing the concentration of the molecules to prevent precipitation resulted in synthetic nanocarrier formation that lacked detectable amounts of one of the molecules.

According to one aspect of the invention, a method is provided for avoiding the interaction of certain molecules in the process of synthetic nanocarrier formation using emulsions. The method involves forming a first emulsion by combining a first fluid containing a first species with a second fluid that is immiscible with the first fluid under conditions to create the first emulsion, wherein the second fluid is the continuous phase of the first emulsion. A second emulsion is formed by combining a third fluid containing a second species with a fourth fluid that is immiscible with the third fluid under conditions to create the second emulsion, wherein the fourth fluid is the continuous phase of the second emulsion. In this method, one or both of the second fluid and the fourth fluid contain particle forming material, and the second fluid and the fourth fluid are miscible. A third emulsion is formed by combining the first emulsion, the second emulsion and a fifth fluid that is immiscible with the second fluid and the fourth fluid under conditions to create the third emulsion, wherein the fifth fluid is the continuous phase of the third emulsion. This third emulsion is a double emulsion of the invention.

In one aspect of the invention, the double emulsion is used in the formation of synthetic nanocarriers. At least a portion of one or both of the second fluid and the fourth fluid is extracted to form synthetic nanocarriers containing the first species and the second species.

The methods of the invention can be practiced with a variety of fluids, a variety of species, a variety of emulsifiers, and a variety of particle forming materials. In one embodiment, the first fluid, just prior to contact with the second fluid to form the first emulsion, is substantially free of the second species, and the third fluid, just prior to contact with the fourth fluid to form the second emulsion, is substantially free of the first species. In one embodiment, the first and second species are such that the first species interacts with the second species to form a precipitate in a solution of either one, both or a mixture of the first and third fluids.

In some embodiments, the third emulsion is formed by combining the first and second emulsions to form a mixture and then combining the mixture with the fifth fluid to form the third emulsion. In some embodiments, the first fluid and the third fluid are miscible. In some embodiments, the first, third and fifth fluids are aqueous. In some embodiments, the first, third and fifth fluids are non-aqueous. In some embodiments the first and the third fluids are identical. In some embodiments the first and the third fluids are not identical. In some embodiments, the second and the fourth fluids are identical. In some embodiments, the second and the fourth fluids are not identical.

In some embodiments, one or both of the first and the second species are pharmaceutical agents. In some embodiments, one or both of the first and the second species are immune modulating agents. An immune modulating agent can be an adjuvant or an antigen. In some embodiments, the first or the second species is an adjuvant. In some embodiments, the first or the second species is an antigen, such as a B-cell antigen or a T cell antigen. In some embodiments, the first or the second species is an oligonucleotide. In some embodiments, the first or the second species is a peptide. In some embodiments, the first species is an adjuvant and the second species is an antigen. In some embodiments, the first species is an oligonucleotide and the second species is a peptide. In one important embodiment, the first species is an immunostimulatory oligonucleotide and the second species is a universal T cell antigen. In one important embodiment, the first species is an immunostimulatory CG containing oligonucleotide, wherein the C of the CG is unmethylated, and the second species is a universal T cell antigen.

In any of the foregoing embodiments, each of the first emulsion, the second emulsion and the third emulsion contain an emulsifier. In any of the forgoing embodiments, the emulsifier of at least the first and second emulsions can comprise the particle forming material. In any of the foregoing embodiments, the fifth fluid can contain an emulsifier.

In any of the foregoing embodiments, the particle forming material can be a polymer. In any of the foregoing embodiments, the particle forming material can be a biodegradable polymer. In any of the foregoing embodiments, the particle forming material can be a biodegradable polymer with a hydrophobic portion and a hydrophilic portion. In any of the foregoing embodiments, the particle forming material can be an amphiphilic biodegradable polymer, including a B cell antigen or a targeting moiety defining a hydrophilic portion. In any of the foregoing embodiments, the particle forming material can comprise PLA or PLGA.

In some embodiments, the first species is dissolved in the first fluid. In some embodiments, the first species is dispersed in the first fluid. In some embodiments, the first species is mixed in the first fluid. In some embodiments, the second species is dissolved in the third fluid. In some embodiments, the second species is dispersed in the third fluid. In some embodiments, the second species is mixed in the third fluid. In one embodiment, the first species is an oligonucleotide dissolved in the first fluid which is aqueous, the second species is a peptide that is a T cell antigen dissolved in the third fluid which is aqueous, and the particle forming material is an amphiphilic biodegradable polymer, including a B cell antigen defining a hydrophilic portion.

The synthetic nanocarriers formed upon extraction of one or both of at least a portion of the second and fourth fluids can have an average size typically of between 100 and 1500 nanometers. Other sizes are possible. More typically, the synthetic nanocarriers formed have a size between about 200 and 1100 nanometers, the synthetic nanocarriers formed have a size between about 200 and 500 nanometers, and in one embodiment, the synthetic nanocarriers have a size between about 200 and 250 nanometers.

According to another aspect of the invention, a product is provided. The product is synthetic nanocarriers formed by any of the methods described above or in greater detail below.

According to another aspect of the invention, a pharmaceutical product is provided. The pharmaceutical product is synthetic nanocarriers formed by any of the methods described above together with a pharmaceutically acceptable carrier. The pharmaceutical product is for administration to a subject. In one aspect, the product is a vaccine to induce an immune response to an antigen.

Other advantages and novel features of the present invention will become apparent from the following detailed description of various non-limiting embodiments of the invention when considered in conjunction with the accompanying figures. In cases where the present specification and a document incorporated by reference include conflicting and/or inconsistent disclosure, the present specification shall control. If two or more documents incorporated by reference include conflicting and/or inconsistent disclosure with respect to each other, then the document having the later effective date shall control.

### A. BRIEF DESCRIPTION OF DRAWINGS

Non-limiting embodiments of the present invention will be described by way of example with reference to the accompanying figures, which are schematic and are not intended to be drawn to scale. In the figures, each identical or nearly identical component illustrated is typically represented by a single numeral. For purposes of clarity, not every component is labeled in every figure, nor is every component of each embodiment of the invention shown where illustration is not necessary to allow those of ordinary skill in the art to understand the invention.

FIGS. 1A-1C illustrate various emulsions in accordance with certain embodiments of the invention; and

FIG. 2 illustrates a method of making an emulsion in accordance with certain embodiments of the invention.

### B. DEFINITIONS

Before describing the present invention in detail, it is to be understood that this invention is not limited to particularly exemplified materials or process parameters as such may, of course, vary. It is also to be understood that the terminology used herein is for the purpose of describing particular embodiments of the invention only, and is not intended to be limiting of the use of alternative terminology to describe the present invention.

All publications, patents and patent applications cited herein, whether supra or infra, are hereby incorporated by reference in their entirety for all purposes.

All definitions, as defined and used herein, should be understood to control over dictionary definitions, definitions in documents incorporated by reference, and/or ordinary meanings of the defined terms, to the extent they are inconsistent with the same.

“Adjuvant” mean an agent that in the context of the invention does not constitute a specific antigen, but modulates the immune response to an antigen (where exposure to the antigen is passive or active).

Adjuvants useful in the present invention include immunostimulatory oligonucleotides, which typically are between 8 and 100 nucleotides long and include one or more 5′-3′ CGs, wherein the C is unmethylated. Immunostimulatory oligonucleotides according to the invention can have a nucleotide sequence that comprises: 5′ AACGTT 3′, 5′ TTCGAA 3′, 5′ GACGTC 3′, 5′ ATCGAT 3′, or 5′ GTCGAC 3′; or in another embodiment have a sequence that comprises 5′ AACGTT 3′, 5′ TTCGAA 3′, 5′ GACGTC 3′, 5′ ATCGAT 3′, 5′ GTCGAC 3′, or 5′ GTCGTT 3′. Other adjuvants useful in the present invention are agonists for toll-like receptors (TLRs) 7 & 8 (“TLR 7/8 agonists”). Of utility are the TLR 7/8 agonist compounds disclosed in U.S. Pat. No. 6,696,076 to Tomai et al., including but not limited to imidazoquinoline amines, imidazopyridine amines, 6,7-fused cycloalkylimidazopyridine amines, and 1,2-bridged imidazoquinoline amines. Preferred adjuvants comprise imiquimod and resiquimod (also known as R848). In specific embodiments, an adjuvant may be an agonist for the DC surface molecule CD40. In certain embodiments, to stimulate immunity rather than tolerance, a synthetic nanocarrier incorporates an adjuvant that promotes DC maturation (needed for priming of naive T cells) and the production of cytokines, such as type I interferons, which promote antibody responses and anti-viral immunity. In some embodiments, an adjuvant may be a TLR-4 agonist, such as bacterial lipopolysacharide (LPS), VSV-G, and/or HMGB-1. In some embodiments, adjuvants may comprise TLR-5 agonists, such as flagellin, or portions or derivatives thereof, including but not limited to those disclosed in U.S. Pat. Nos. 6,130,082, 6,585,980, and 7,192,725. In some embodiments, adjuvants may be proinflammatory stimuli released from necrotic cells (e.g., urate crystals). In some embodiments, adjuvants may be activated components of the complement cascade (e.g., CD21, CD35, etc.). In some embodiments, adjuvants may be activated components of immune complexes. The adjuvants also include complement receptor agonists, such as a molecule that binds to CD21 or CD35. In some embodiments, the complement receptor agonist induces endogenous complement opsonization of the synthetic nanocarrier. In some embodiments, adjuvants are cytokines, which are small proteins or biological factors (in the range of 5 kD-20 kD) that are released by cells and have specific effects on cell-cell interaction, communication and behavior of other cells. In some embodiments, the cytokine receptor agonist is a small molecule, antibody, fusion protein, or aptamer.

“Administering” or “administration” means providing a drug to a subject in a manner that is pharmacologically useful.

“Antigen” means an agent that in the context of the invention constitutes a B cell antigen or T cell antigen.

“APC targeting feature” means one or more portions of which the inventive synthetic nanocarriers are comprised that target the synthetic nanocarriers to professional antigen presenting cells (“APCs”), such as but not limited to dendritic cells, SCS macrophages, follicular dendritic cells, and B cells.

In embodiments, targeting moieties for known targets on macrophages (“Mphs”) comprise any targeting moiety that specifically binds to any entity (e.g., protein, lipid, carbohydrate, small molecule, etc.) that is prominently expressed and/or present on macrophages (i.e., subcapsular sinus-Mph markers). Exemplary SCS-Mph markers include, but are not limited to, CD4 (L3T4, W3/25, T4); CD9 (p24, DRAP-1, MRP-1); CD11a (LFA-1α, α L Integrin chain); CD11b (αM Integrin chain, CR3, Mo1, C3niR, Mac-1); CD11c (αX Integrin, p150, 95, AXb2); CDw12 (p90-120); CD13 (APN, gp150, EC 3.4.11.2); CD14 (LPS-R); CD15 (X-Hapten, Lewis, X, SSEA-1,3-FAL); CD15s (Sialyl Lewis X); CD15u (3′ sulpho Lewis X); CD15su (6 sulpho-sialyl Lewis X); CD16a (FCRIIIA); CD16b (FcgRIIIb); CDw17 (Lactosylceramide, LacCer); CD18 (Integrin (β2, CD11a,b,c β-subunit); CD26 (DPP IV ectoeneyme, ADA binding protein); CD29 (Platelet GPIIa, β-1 integrin, GP); CD31 (PECAM-1, Endocam); CD32 (FCγRII); CD33 (gp67); CD35 (CR1, C3b/C4b receptor); CD36 (GpIIIb, GPIV, PASIV); CD37 (gp52-40); CD38 (ADP-ribosyl cyclase, T10); CD39 (ATPdehydrogenase, NTPdehydrogenase-1); CD40 (Bp50); CD43 (Sialophorin, Leukosialin); CD44 (EMCRII, H-CAM, Pgp-1); CD45 (LCA, T200, B220, Ly5); CD45RA; CD45RB; CD45RC; CD45RO (UCHL-1); CD46 (MCP); CD47 (gp42, IAP, OA3, Neurophillin); CD47R (MEM-133); CD48 (Blast-1, Hulym3, BCM-1, OX-45); CD49a (VLA-1α, α1 Integrin); CD49b (VLA-2α, gpla, α2 Integrin); CD49c (VLA-3α, α3 Integrin); CD49e (VLA-5α, α5 Integrin); CD49f (VLA-6α, α6 Integrin, gplc); CD50 (ICAM-3); CD51 (Integrin α, VNR-α, Vitronectin-Rα); CD52 (CAMPATH-1, HE5); CD53 (OX-44); CD54 (ICAM-1); CD55 (DAF); CD58 (LFA-3); CD59 (1F5Ag, H19, Protectin, MACIF, MIRL, P-18); CD60a (GD3); CD60b (9-O-acetyl GD3); CD61 (GP IIIa, β3 Integrin); CD62L (L-selectin, LAM-1, LECAM-1, MEL-14, Leu8, TQ1); CD63 (LIMP, MLA1, gp55, NGA, LAMP-3, ME491); CD64 (FcγRI); CD65 (Ceramide, VIM-2); CD65s (Sialylated-CD65, VIM2); CD72 (Ly-19.2, Ly-32.2, Lyb-2); CD74 (Ii, invariant chain); CD75 (sialo-masked Lactosamine); CD75S (α-2,6 sialylated Lactosamine); CD80 (B7, B7-1, BB1); CD81 (TAPA-1); CD82 (4F9, C33, IA4, KAI1, R2); CD84 (p75, GR6); CD85a (ILT5, LIR2, HL9); CD85d (ILT4, LIR2, MIR10); CD85j (ILT2, LIR1, MIR7); CD85k (ILT3, LIR5, HM18); CD86 (B7-2/B70); CD87 (uPAR); CD88 (C5aR); CD89 (IgA Fc receptor, FcαR); CD91 (α2M-R, LRP); CDw92 (p70); CDw93 (GR11); CD95 (APO-1, FAS, TNFRSF6); CD97 (BL-KDD/F12); CD98 (4F2, FRP-1, RL-388); CD99 (MIC2, E2); CD99R (CD99 Mab restricted); CD100 (SEMA4D); CD101 (IGSF2, P126, V7); CD102 (ICAM-2); CD111 (PVRL1, HveC, PRR1, Nectin 1, HlgR); CD112 (HveB, PRR2, PVRL2, Nectin2); CD114 (CSF3R, G-CSRF, HG-CSFR); CD115 (c-fms, CSF-1R, M-CSFR); CD116 (GMCSFRα); CDw119 (IFNγR, IFNγRA); CD120a (TNFRI, p55); CD120b (TNFRII, p75, TNFR p80); CD121b (Type 2 IL-1R); CD122 (IL2Rβ); CD123 (IL-3Rα); CD124 (IL-4Rα); CD127 (p90, IL-7R, IL-7Rα); CD128a (IL-8Rα, CXCR1, (Tentatively renamed as CD181)); CD128b (IL-8Rb, CSCR2, (Tentatively renamed as CD182)); CD130 (gp130); CD131 (Common β subunit); CD132 (Common γ chain, IL-2Rγ); CDw136 (MSP-R, RON, p158-ron); CDw137 (4-1BB, ILA); CD139; CD141 (Thrombomodulin, Fetomodulin); CD147 (Basigin, EMMPRIN, M6, OX47); CD148 (HPTP-η, p260, DEP-1); CD155 (PVR); CD156a (CD156, ADAMS, MS2); CD156b (TACE, ADAM17, cSVP); CDw156C (ADAM10); CD157 (Mo5, BST-1); CD162 (PSGL-1); CD164 (MGC-24, MUC-24); CD165 (AD2, gp37); CD168 (RHAMM, IHABP, HMMR); CD169 (Sialoadhesin, Siglec-1); CD170 (Siglec 5); CD171 (L1CAM, NILE); CD172 (SIRP-1α, MyD-1); CD172b (SIRPβ); CD180 (RP105, Bgp95, Ly64); CD181 (CXCR1, (Formerly known as CD128a)); CD182 (CXCR2, (Formerly known as CD128b)); CD184 (CXCR4, NPY3R); CD191 (CCR1); CD192 (CCR2); CD195 (CCR5); CDw197 (CCR7 (was CDw197)); CDw198 (CCR8); CD204 (MSR); CD205 (DEC-25); CD206 (MMR); CD207 (Langerin); CDw210 (CK); CD213a (CK); CDw217 (CK); CD220 (Insulin R); CD221 (IGF1 R); CD222 (M6P-R, IGFII-R); CD224 (GGT); CD226 (DNAM-1, PTA1); CD230 (Prion Protein (PrP)); CD232 (VESP-R); CD244 (2B4, P38, NAIL); CD245 (p220/240); CD256 (APRIL, TALL2, TNF (ligand) superfamily, member 13); CD257 (BLYS, TALL1, TNF (ligand) superfamily, member 13b); CD261 (TRAIL-R1, TNF-R superfamily, member 10a); CD262 (TRAIL-R2, TNF-R superfamily, member 10b); CD263 (TRAIL-R3, TNBF-R superfamily, member 10c); CD264 (TRAIL-R4, TNF-R superfamily, member 10d); CD265 (TRANCE-R, TNF-R superfamily, member 11a); CD277 (BT3.1, B7 family: Butyrophilin 3); CD280 (TEM22, ENDO180); CD281 (TLR1, TOLL-like receptor 1); CD282 (TLR2, TOLL-like receptor 2); CD284 (TLR4, TOLL-like receptor 4); CD295 (LEPR); CD298 (ATP1B3, Na K ATPase, β3 subunit); CD300a (CMRF-35H); CD300c (CMRF-35A); CD300e (CMRF-35L1); CD302 (DCL1); CD305 (LAIR1); CD312 (EMR2); CD315 (CD9P1); CD317 (BST2); CD321 (JAM1); CD322 (JAM2); CDw328 (Siglec7); CDw329 (Siglec9); CD68 (gp 110, Macrosialin); and/or mannose receptor; wherein the names listed in parentheses represent alternative names.

In embodiments, targeting moieties for known targets on dendritic cells (“DCs”) comprise any targeting moiety that specifically binds to any entity (e.g., protein, lipid, carbohydrate, small molecule, etc.) that is prominently expressed and/or present on DCs (i.e., a DC marker). Exemplary DC markers include, but are not limited to, CD1a (R4, T6, HTA-1); CD1b (R1); CD1c (M241, R7); CD1d (R3); CD1e (R2); CD11b (αM Integrin chain, CR3, Mo1, C3niR, Mac-1); CD11c (αX Integrin, p150, 95, AXb2); CDw117 (Lactosylceramide, LacCer); CD19 (B4); CD33 (gp67); CD 35 (CR1, C3b/C4b receptor); CD 36 (GpIIIb, GPIV, PASIV); CD39 (ATPdehydrogenase, NTPdehydrogenase-1); CD40 (Bp50); CD45 (LCA, T200, B220, Ly5); CD45RA; CD45RB; CD45RC; CD45RO (UCHL-1); CD49d (VLA-4α, α4 Integrin); CD49e (VLA-5α, α5 Integrin); CD58 (LFA-3); CD64 (FcγRI); CD72 (Ly-19.2, Ly-32.2, Lyb-2); CD73 (Ecto-5′ nucloticlase); CD74 (Ii, invariant chain); CD80 (B7, B7-1, BB1); CD81 (TAPA-1); CD83 (HB15); CD85a (ILT5, LIR3, HL9); CD85d (ILT4, LIR2, MIR10); CD85j (ILT2, LIR1, MIR7); CD85k (ILT3, LIR5, HM18); CD86 (B7-2/B70); CD88 (C5aB); CD97 (BL-KDD/F12); CD101 (IGSF2, P126, V7); CD116 (GM-CSFRα); CD120a (TMFRI, p55); CD120b (TNFRII, p75, TNFR p80); CD123 (IL-3Rα); CD139; CD148 (HPTP-η, DEP-1); CD150 (SLAM, IPO-3); CD156b (TACE, ADAM17, cSVP); CD157 (Mo5, BST-1); CD167a (DDR1, trkE, cak); CD168 (RHAMM, IHABP, HMMR); CD169 (Sialoadhesin, Siglec-1); CD170 (Siglec-5); CD171 (L1CAM, NILE); CD172 (SIRP-1α, MyD-1); CD172b (SIRPβ); CD180 (RP105, Bgp95, Ly64); CD184 (CXCR4, NPY3R); CD193 (CCR3); CD196 (CCR6); CD197 (CCR7 (ws CDw197)); CDw197 (CCR7, EBI1, BLR2); CD200 (OX2); CD205 (DEC-205); CD206 (MMR); CD207 (Langerin); CD208 (DC-LAMP); CD209 (DCSIGN); CDw218a (IL18Rα); CDw218b (IL8Rβ); CD227 (MUC1, PUM, PEM, EMA); CD230 (Prion Protein (PrP)); CD252 (OX40L, TNF (ligand) superfamily, member 4); CD258 (LIGHT, TNF (ligand) superfamily, member 14); CD265 (TRANCE-R, TNF-R superfamily, member 11a); CD271 (NGFR, p75, TNFR superfamily, member 16); CD273 (B7DC, PDL2); CD274 (B7H1, PDL1); CD275 (B7H2, ICOSL); CD276 (B7H3); CD277 (BT3.1, B7 family: Butyrophilin 3); CD283 (TLR3, TOLL-like receptor 3); CD289 (TLR9, TOLL-like receptor 9); CD295 (LEPR); CD298 (ATP1B3, Na K ATPase β3 submit); CD300a (CMRF-35H); CD300c (CMRF-35A); CD301 (MGL1, CLECSF14); CD302 (DCL1); CD303 (BDCA2); CD304 (BDCA4); CD312 (EMR2); CD317 (BST2); CD319 (CRACC, SLAMF7); CD320 (8D6); and CD68 (gp110, Macrosialin); class II MHC; BDCA-1; Siglec-H; wherein the names listed in parentheses represent alternative names.

In embodiments, targeting can be accomplished by any targeting moiety that specifically binds to any entity (e.g., protein, lipid, carbohydrate, small molecule, etc.) that is prominently expressed and/or present on B cells (i.e., B cell marker). Exemplary B cell markers include, but are not limited to, CD1c (M241, R7); CD1d (R3); CD2 (E-rosette R, T11, LFA-2); CD5 (T1, Tp67, Leu-1, Ly-1); CD6 (T12); CD9 (p24, DRAP-1, MRP-1); CD11a (LFA-1α, αL Integrin chain); CD11b (αM Integrin chain, CR3, Mo1, C3niR, Mac-1); CD11c (αX Integrin, P150, 95, AXb2); CDw17 (Lactosylceramide, LacCer); CD18 (Integrin β2, CD11a, b, c β-subunit); CD19 (B4); CD20 (B1, Bp35); CD21 (CR2, EBV-R, C3dR); CD22 (BL-CAM, Lyb8, Siglec-2); CD23 (FceRII, B6, BLAST-2, Leu-20); CD24 (BBA-1, HSA); CD25 (Tac antigen, IL-2Rα, p55); CD26 (DPP IV ectoeneyme, ADA binding protein); CD27 (T14, S152); CD29 (Platelet GPIIa, β-1 integrin, GP); CD31 (PECAM-1, Endocam); CD32 (FCγRII); CD35 (CR1, C3b/C4b receptor); CD37 (gp52-40); CD38 (ADPribosyl cyclase, T10); CD39 (ATPdehydrogenase, NTPdehydrogenase-1); CD40 (Bp50); CD44 (ECMRII, H-CAM, Pgp-1); CD45 (LCA, T200, B220, Ly5); CD45RA; CD45RB; CD45RC; CD45RO (UCHL-1); CD46 (MCP); CD47 (gp42, IAP, OA3, Neurophilin); CD47R (MEM-133); CD48 (Blast-1, Hulym3, BCM-1, OX-45); CD49b (VLA-2α, gpla, α2 Integrin); CD49c (VLA-3α, α3 Integrin); CD49d (VLA-4α, α4 Integrin); CD50 (ICAM-3); CD52 (CAMPATH-1, HES); CD53 (OX-44); CD54 (ICAM-1); CD55 (DAF); CD58 (LFA-3); CD60a (GD3); CD62L (L-selectin, LAM-1, LECAM-1, MEL-14, Leu8, TQ1); CD72 (Ly-19.2, Ly-32.2, Lyb-2); CD73 (Ecto-5′-nuciotidase); CD74 (Ii, invariant chain); CD75 (sialo-masked Lactosamine); CD75S (α2, 6 sialylated Lactosamine); CD77 (Pk antigen, BLA, CTH/Gb3); CD79a (Igα, MB1); CD79b (IGβ, B29); CD80; CD81 (TAPA-1); CD82 (4F9, C33, IA4, KAI1, R2); CD83 (HB15); CD84 (P75, GR6); CD85j (ILT2, LIR1, MIR7); CDw92 (p70); CD95 (APO-1, FAS, TNFRSF6); CD98 (4F2, FRP-1, RL-388); CD99 (MIC2, E2); CD100 (SEMA4D); CD102 (ICAM-2); CD108 (SEMA7A, JMH blood group antigen); CDw119 (IFNγR, IFNγRa); CD120a (TNFRI, p55); CD120b (TNFRII, p75, TNFR p80); CD121b (Type 2 IL-1R); CD122 (IL2Rβ); CD124 (IL-4Rα); CD130 (gp130); CD132 (Common γ chain, IL-2Rγ); CDw137 (4-1 BB, ILA); CD139; CD147 (Basigin, EMMPRIN, M6, OX47); CD150 (SLAM, IPO-3); CD162 (PSGL-1); CD164 (MGC-24, MUC-24); CD166 (ALCAM, KG-CAM, SC-1, BEN, DM-GRASP); CD167a (DDR1, trkE, cak); CD171 (L1CMA, NILE); CD175s (Sialyl-Tn (S-Tn)); CD180 (RP105, Bgp95, Ly64); CD184 (CXCR4, NPY3R); CD185 (CXCR5); CD192 (CCR2); CD196 (CCR6); CD197 (CCR7 (was CDw197)); CDw197 (CCR7, EBI1, BLR2); CD200 (OX2); CD205 (DEC-205); CDw210 (CK); CD213a (CK); CDw217 (CK); CDw218a (IL18Rα); CDw218b (IL18Rβ); CD220 (Insulin R); CD221 (IGF1R); CD222 (M6P-R, IGFII-R); CD224 (GGT); CD225 (Leu13); CD226 (DNAM-1, PTA1); CD227 (MUC1, PUM, PEM, EMA); CD229 (Ly9); CD230 (Prion Protein (Prp)); CD232 (VESP-R); CD245 (p220/240); CD247 (CD3 Zeta Chain); CD261 (TRAIL-R1, TNF-R superfamily, member 10a); CD262 (TRAIL-R2, TNF-R superfamily, member 10b); CD263 (TRAIL-R3, TNF-R superfamily, member 10c); CD264 (TRAIL-R4, TNF-R superfamily, member 10d); CD265 (TRANCE-R TNF-R superfamily, member 11a); CD267 (TACI, TNF-R superfamily, member 13B); CD268 (BAFFR, TNF-R superfamily, member 13C); CD269 (BCMA, TNF-R superfamily, member 16); CD275 (B7H2, ICOSL); CD277 (BT3.1.B7 family: Butyrophilin 3); CD295 (LEPR); CD298 (ATP1B3 Na K ATPase β3 subunit); CD300a (CMRF-35H); CD300c (CMRF-35A); CD305 (LAIR1); CD307 (IRTA2); CD315 (CD9P1); CD316 (EW12); CD317 (BST2); CD319 (CRACC, SLAMF7); CD321 (JAM1); CD322 (JAM2); CDw327 (Siglec6, CD33L); CD68 (gp 100, Macrosialin); CXCR5; VLA-4; class II MHC; surface IgM; surface IgD; APRL; and/or BAFF-R; wherein the names listed in parentheses represent alternative names. Examples of markers include those provided elsewhere herein.

In some embodiments, B cell targeting can be accomplished by any targeting moiety that specifically binds to any entity (e.g., protein, lipid, carbohydrate, small molecule, etc.) that is prominently expressed and/or present on B cells upon activation (i.e., activated B cell marker). Exemplary activated B cell markers include, but are not limited to, CD1a (R4, T6, HTA-1); CD1b (R1); CD15s (Sialyl Lewis X); CD15u (3′ sulpho Lewis X); CD15su (6 sulpho-sialyl Lewis X); CD30 (Ber-H2, Ki-1); CD69 (AIM, EA 1, MLR3, gp34/28, VEA); CD70 (Ki-24, CD27 ligand); CD80 (B7, B7-1, BB1); CD86 (B7-2/B70); CD97 (BLKDD/F12); CD125 (IL-5Rα); CD126 (IL-6Rα); CD138 (Syndecan-1, Heparan sulfate proteoglycan); CD152 (CTLA-4); CD252 (OX40L, TNF(ligand) superfamily, member 4); CD253 (TRAIL, TNF(ligand) superfamily, member 10); CD279 (PD1); CD289 (TLR9, TOLL-like receptor 9); and CD312 (EMR2); wherein the names listed in parentheses represent alternative names. Examples of markers include those provided elsewhere herein.

“B cell antigen” means any antigen that is recognized by and triggers an immune response in a B cell (e.g., an antigen that is specifically recognized by a B cell receptor on a B cell). In some embodiments, an antigen that is a T cell antigen is also a B cell antigen. In other embodiments, the T cell antigen is not also a B cell antigen.

B cell antigens include, but are not limited to proteins, peptides, glycoproteins, small molecules, and carbohydrates. In some embodiments, the B cell antigen is a non-protein antigen (i.e., not a protein or peptide antigen). In some embodiments, the B cell antigen is a carbohydrate associated with an infectious agent. In some embodiments, the B cell antigen is a glycoprotein or glycopeptide associated with an infectious agent. The infectious agent can be a bacterium, virus, fungus, protozoan, or parasite. In some embodiments, the B cell antigen is a poorly immunogenic antigen.

In some embodiments, the B cell antigen is an abused substance or a portion thereof. In some embodiments, the B cell antigen is an addictive substance or a portion thereof. Addictive substances include, but are not limited to, nicotine, a narcotic, a cough suppressant, a tranquilizer, and a sedative. In some embodiments, the B cell antigen is a toxin, such as a toxin from a chemical weapon or natural sources. The B cell antigen may also be a hazardous environmental agent. In some embodiments, the B cell antigen is a self antigen. In other embodiments, the B cell antigen is an alloantigen, an allergen, a contact sensitizer, a degenerative disease antigen, a hapten, an infectious disease antigen, a cancer antigen, an atopic disease antigen, an autoimmune disease antigen, an addictive substance, a xenoantigen, or a metabolic disease enzyme or enzymatic product thereof.

“Couple” or “Coupled” or “Couples” (and the like) means to chemically associate one entity (for example a moiety) with another. In some embodiments, the coupling is covalent. In some embodiments, the coupling is non-covalent. In non-covalent embodiments, the non-covalent coupling is mediated by interactions comprising charge interactions, affinity interactions, metal coordination, physical adsorption, host-guest interactions, hydrophobic interactions, TT stacking interactions, hydrogen bonding interactions, van der Waals interactions, magnetic interactions, electrostatic interactions, dipole-dipole interactions, and/or combinations thereof.

“Encapsulate” means to enclose within a synthetic nanocarrier, in some embodiments completely within a synthetic nanocarrier. Most or all of a substance that is encapsulated is not exposed to the local environment external to the synthetic nanocarrier prior to degradation if the nanocarrier is biodegradable. Encapsulation is distinct from absorption, which places most or all of a substance on a surface of a synthetic nanocarrier, and leaves the substance exposed to the local environment external to the synthetic nanocarrier.

“Emulsifier” means an agent that stabilizes an emulsion. Emulsifiers are well known and the selection will depend on the fluids selected for the emulsion as well as which fluid is to be the continuous phase. Emulsifiers include surfactants. Commonly used surfactants include cetyltrimethylammonium bromide (CTAB), benzalkonium chloride, dimethyl dioctodecyl ammonium bromide (DDA), dioleoyl-3-trimethylammonium-propane (DOTAP), Sodium cholate, sodium dodecyl sulfate (SDS)/sodium lauryl sulfate (SLS), disulfosuccinate (DSS), sulphated fatty alcohols, polyvinyl alcohol (PVA), polyvinylpyrrolidone (PVP), sorbitan esters polysorbates, polyoxyethylated glycol monoethers, polyoxyethylated alkyl phenols and poloxamers. Particle forming material, described in general below, also can act as an emulsifier.

“Oligonucleotide” means a nucleotide molecule having from 6 to 100 nucleotides, preferably from 8 to 75 nucleotides, and more typically from 10 to 50 nucleotides, still more typically from 15 to 25 nucleotides. In an embodiment according to the invention, oligonucleotides comprise less than 100 nucleotides, less than 50 nucleotides, less than 25 nucleotides, and even less than 10 nucleotides. In embodiments, oligonucleotides according to the invention possess a phosphodiester backbone that is not modified or possess a backbone that is modified, for example to incorporate phosphorothioate bonds. In some embodiments, the backbone is free of phosphorothioate bonds. In some embodiments, the oligonucleotides' phosphodiester backbone comprises no stabilizing chemical modifications that function to stabilize the phosphodiester backbone under physiological conditions. In other embodiments, the oligonucleotides' comprises stabilizing chemical modifications that function to stabilize the backbone under physiological conditions. Thus, oligonucleotides can comprise nucleoside analogs such as analogs having chemically modified bases or sugars, backbone modifications, etc. In some embodiments, an oligonucleotide is or comprises natural nucleosides (e.g., adenosine, thymidine, guanosine, cytidine, uridine, deoxyadenosine, deoxythymidine, deoxyguanosine, and deoxycytidine); nucleoside analogs (e.g., 2-aminoadenosine, 2-thiothymidine, inosine, pyrrolo-pyrimidine, 3-methyl adenosine, 5-methylcytidine, C-5 propynyl-cytidine, C-5 propynyl-uridine, 2-aminoadenosine, C5-bromouridine, C5-fluorouridine, C5-iodouridine, C5-propynyl-uridine, C5-propynylcytidine, C5-methylcytidine, 2-aminoadenosine, 7-deazaadenosine, 7-deazaguanosine, 8-oxoadenosine, 8-oxoguanosine, O(6)-methylguanine, and 2-thiocytidine); chemically modified bases; biologically modified bases (e.g., methylated bases); intercalated bases; modified sugars (e.g., 2′-fluororibose, ribose, 2′-deoxyribose, arabinose, and hexose); and/or modified phosphate groups (e.g., phosphorothioates and 5′-N-phosphoramidite linkages).

“Particle forming material” means a material of which the formed synthetic nanocarrier is comprised upon extraction of the second and/or fourth fluids. Typically the particle forming material is a polymer, natural or synthetic.

A wide variety of polymers may be used as particle forming material in the formation of synthetic nanocarriers for biological use. In general, polymers may be homopolymers or copolymers comprising two or more monomers. In terms of sequence, copolymers may be random, block, or comprise a combination of random and block sequences. Typically, polymers in accordance with the present invention are organic polymers.

Examples of polymers suitable for use in the present invention include, but are not limited to polyethylenes, polycarbonates (e.g. poly(1,3-dioxan-2one)), polyanhydrides (e.g. poly(sebacic anhydride)), polyhydroxyacids (e.g. poly(β-hydroxyalkanoate)), polypropylfumerates, polycaprolactones, polyamides (e.g. polycaprolactam), polyacetals, polyethers, polyesters (e.g., polylactide, polyglycolide), poly(orthoesters), polycyanoacrylates, polyvinyl alcohols, polyurethanes, polyphosphazenes, polyacrylates, polymethacrylates, polyureas, polystyrenes, and polyamines. In some embodiments, the polymer is polylysine, polylysine-PEG copolymers, poly(ethyleneimine), poly(ethylene imine)-PEG copolymers.

In some embodiments, polymers in accordance with the present invention include polymers which have been approved for use in humans by the U.S. Food and Drug Administration (FDA) under 21 C.F.R. §177.2600, including but not limited to polyesters (e.g., polylactic acid, poly(lactic-co-glycolic acid), polycaprolactone, polyvalerolactone, poly(1,3-dioxan-2one)); polyanhydrides (e.g., poly(sebacic anhydride)); polyethers (e.g., polyethylene glycol); polyurethanes; polymethacrylates; polyacrylates; and polycyanoacrylates.

In some embodiments, polymers can be hydrophilic. For example, polymers may comprise anionic groups (e.g., phosphate group, sulphate group, carboxylate group); cationic groups (e.g., quaternary amine group); or polar groups (e.g., hydroxyl group, thiol group, amine group). In some embodiments, a synthetic nanocarrier comprising a hydrophilic polymeric matrix generates a hydrophilic environment within the synthetic nanocarrier. In some embodiments, polymers can be hydrophobic. In some embodiments, a synthetic nanocarrier comprising a hydrophobic polymeric matrix generates a hydrophobic environment within the synthetic nanocarrier. Selection of the hydrophilicity or hydrophobicity of the polymer may have an impact on the nature of materials that are incorporated (e.g. coupled) within the synthetic nanocarrier.

In some embodiments, polymers may be modified with one or more moieties and/or functional groups. A variety of moieties or functional groups can be used in accordance with the present invention. In some embodiments, polymers may be modified with polyethylene glycol (PEG), with a carbohydrate, and/or with acyclic polyacetals derived from polysaccharides (Papisov, 2001, ACS Symposium Series, 786:301). Certain embodiments may be made using the general teachings of U.S. Pat. No. 5,543,158 to Gref et al., or WO publication WO2009/051837 by Von Andrian et al.

In some embodiments, polymers may be modified with a lipid or fatty acid group. In some embodiments, a fatty acid group may be one or more of butyric, caproic, caprylic, capric, lauric, myristic, palmitic, stearic, arachidic, behenic, or lignoceric acid. In some embodiments, a fatty acid group may be one or more of palmitoleic, oleic, vaccenic, linoleic, alpha-linoleic, gamma-linoleic, arachidonic, gadoleic, arachidonic, eicosapentaenoic, docosahexaenoic, or erucic acid.

In some embodiments, polymers may be polyesters, including copolymers comprising lactic acid and glycolic acid units, such as poly(lactic acid-co-glycolic acid) and poly(lactide-co-glycolide), collectively referred to herein as “PLGA”; and homopolymers comprising glycolic acid units, referred to herein as “PGA,” and lactic acid units, such as poly-L-lactic acid, poly-D-lactic acid, poly-D,L-lactic acid, poly-L-lactide, poly-D-lactide, and poly-D,L-lactide, collectively referred to herein as “PLA.” In some embodiments, exemplary polyesters include, for example, polyhydroxyacids; PEG copolymers and copolymers of lactide and glycolide (e.g., PLA-PEG copolymers, PGA-PEG copolymers, PLGA-PEG copolymers, and derivatives thereof. In some embodiments, polyesters include, for example, polyanhydrides, poly(ortho ester), poly(ortho ester)-PEG copolymers, poly(caprolactone), poly(caprolactone)-PEG copolymers, poly(L-lactide-co-L-lysine), poly(serine ester), poly(4-hydroxy-L-proline ester), poly[α-(4-aminobutyl)-L-glycolic acid], and derivatives thereof.

In some embodiments, a polymer may be PLGA. PLGA is a biocompatible and biodegradable co-polymer of lactic acid and glycolic acid, and various forms of PLGA are characterized by the ratio of lactic acid:glycolic acid. Lactic acid can be L-lactic acid, D-lactic acid, or D,L-lactic acid. The degradation rate of PLGA can be adjusted by altering the lactic acid:glycolic acid ratio. In some embodiments, PLGA to be used in accordance with the present invention is characterized by a lactic acid:glycolic acid ratio of approximately 85:15, approximately 75:25, approximately 60:40, approximately 50:50, approximately 40:60, approximately 25:75, or approximately 15:85.

In some embodiments, polymers may be one or more acrylic polymers. In certain embodiments, acrylic polymers include, for example, acrylic acid and methacrylic acid copolymers, methyl methacrylate copolymers, ethoxyethyl methacrylates, cyanoethyl methacrylate, aminoalkyl methacrylate copolymer, poly(acrylic acid), poly(methacrylic acid), methacrylic acid alkylamide copolymer, poly(methyl methacrylate), poly(methacrylic acid anhydride), methyl methacrylate, polymethacrylate, poly(methyl methacrylate) copolymer, polyacrylamide, aminoalkyl methacrylate copolymer, glycidyl methacrylate copolymers, polycyanoacrylates, and combinations comprising one or more of the foregoing polymers. The acrylic polymer may comprise fully-polymerized copolymers of acrylic and methacrylic acid esters with a low content of quaternary ammonium groups.

In some embodiments, polymers can be cationic polymers. In general, cationic polymers are able to condense and/or protect negatively charged strands of nucleic acids (e.g. DNA, or derivatives thereof). Amine-containing polymers such as poly(lysine) (Zauner et al., 1998, Adv. Drug Del. Rev., 30:97; and Kabanov et al., 1995, Bioconjugate Chem., 6:7), poly(ethylene imine) (PEI; Boussif et al., 1995, Proc. Natl. Acad. Sci., USA, 1995, 92:7297), and poly(amidoamine) dendrimers (Kukowska-Latallo et al., 1996, Proc. Natl. Acad. Sci., USA, 93:4897; Tang et al., 1996, Bioconjugate Chem., 7:703; and Haensler et al., 1993, Bioconjugate Chem., 4:372) are positively-charged at physiological pH, form ion pairs with nucleic acids, and mediate transfection in a variety of cell lines. In embodiments, the inventive synthetic nanocarriers may not comprise (or may exclude) cationic polymers.

In some embodiments, polymers can be degradable polyesters bearing cationic side chains (Putnam et al., 1999, Macromolecules, 32:3658; Barrera et al., 1993, J. Am. Chem. Soc., 115:11010; Kwon et al., 1989, Macromolecules, 22:3250; Lim et al., 1999, J. Am. Chem. Soc., 121:5633; and Zhou et al., 1990, Macromolecules, 23:3399). Examples of these polyesters include poly(L-lactide-co-Llysine) (Barrera et al., 1993, J. Am. Chem. Soc., 115:11010), poly(serine ester) (Zhou et al., 1990, Macromolecules, 23:3399), poly(4-hydroxy-L-proline ester) (Putnam et al., 1999, Macromolecules, 32:3658; and Lim et al., 1999, J. Am. Chem. Soc., 121:5633), and poly(4-hydroxy-L-proline ester) (Putnam et al., 1999, Macromolecules, 32:3658; and Lim et al., 1999, J. Am. Chem. Soc., 121:5633).

The properties of these and other polymers and methods for preparing them are well known in the art (see, for example, U.S. Pat. Nos. 6,123,727; 5,804,178; 5,770,417; 5,736,372; 5,716,404; 6,095,148; 5,837,752; 5,902,599; 5,696,175; 5,514,378; 5,512,600; 5,399,665; 5,019,379; 5,010,167; 4,806,621; 4,638,045; and 4,946,929; Wang et al., 2001, J. Am. Chem. Soc., 123:9480; Lim et al., 2001, J. Am. Chem. Soc., 123:2460; Langer, 2000, Acc. Chem. Res., 33:94; Langer, 1999, J. Control. Release, 62:7; and Uhrich et al., 1999, Chem. Rev., 99:3181). More generally, a variety of methods for synthesizing certain suitable polymers are described in Concise Encyclopedia of Polymer Science and Polymeric Amines and Ammonium Salts, Ed. by Goethals, Pergamon Press, 1980; Principles of Polymerization by Odian, John Wiley & Sons, Fourth Edition, 2004; Contemporary Polymer Chemistry by Allcock et al., Prentice-Hall, 1981; Deming et al., 1997, Nature, 390:386; and in U.S. Pat. Nos. 6,506,577, 6,632,922, 6,686,446, and 6,818,732.

In some embodiments, polymers can be linear or branched polymers. In some embodiments, polymers can be dendrimers. In some embodiments, polymers can be substantially cross-linked to one another. In some embodiments, polymers can be substantially free of cross-links. In some embodiments, polymers can be used in accordance with the present invention without undergoing a cross-linking step. It is further to be understood that inventive synthetic nanocarriers may comprise block copolymers, graft copolymers, blends, mixtures, and/or adducts of any of the foregoing and other polymers. Those skilled in the art will recognize that the polymers listed herein represent an exemplary, not comprehensive, list of polymers that can be of use in accordance with the present invention.

In some embodiments, the particle forming material and formed synthetic nanocarriers may comprise a nonpolymeric component. In some embodiments, the particle forming material and the formed synthetic nanocarriers may comprise metal particles, quantum dots, ceramic particles, etc. In some embodiments, a non-polymeric particle forming material results in a synthetic nanocarrier that is an aggregate of non-polymeric components, such as an aggregate of metal atoms (e.g., gold atoms).

In some embodiments, particle forming material and the formed synthetic nanocarrier may optionally comprise one or more amphiphilic entities. In some embodiments, an amphiphilic entity can promote the production of synthetic nanocarriers with increased stability, improved uniformity, or increased viscosity. In some embodiments, amphiphilic entities can be associated with the interior surface of a lipid membrane (e.g., lipid bilayer, lipid monolayer, etc.). Many amphiphilic entities known in the art are suitable for use in making synthetic nanocarriers in accordance with the present invention. Such amphiphilic entities include, but are not limited to, phosphoglycerides; phosphatidylcholines; dipalmitoyl phosphatidylcholine (DPPC); dioleylphosphatidyl ethanolamine (DOPE); dioleyloxypropyltriethylammonium (DOTMA); dioleoylphosphatidylcholine; cholesterol; cholesterol ester; diacylglycerol; diacylglycerolsuccinate; diphosphatidyl glycerol (DPPG); hexanedecanol; fatty alcohols such as polyethylene glycol (PEG); polyoxyethylene-9-lauryl ether; a surface active fatty acid, such as palmitic acid or oleic acid; fatty acids; fatty acid monoglycerides; fatty acid diglycerides; fatty acid amides; sorbitan trioleate (Span®85) glycocholate; sorbitan monolaurate (Span®20); polysorbate 20 (Tween®20); polysorbate 60 (Tween®60); polysorbate 65 (Tween®65); polysorbate 80 (Tween®80); polysorbate 85 (Tween®85); polyoxyethylene monostearate; surfactin; a poloxomer; a sorbitan fatty acid ester such assorbitan trioleate; lecithin; lysolecithin; phosphatidylserine; phosphatidylinositol; sphingomyelin; phosphatidylethanolamine (cephalin); cardiolipin; phosphatidic acid; cerebrosides; dicetylphosphate; dipalmitoylphosphatidylglycerol; stearylamine; dodecylamine; hexadecyl-amine; acetyl palmitate; glycerol ricinoleate; hexadecyl sterate; isopropyl myristate; tyloxapol; poly(ethylene glycol)5000-phosphatidylethanolamine; poly(ethylene glycol)400-monostearate; phospholipids; synthetic and/or natural detergents having high surfactant properties; deoxycholates; cyclodextrins; chaotropic salts; ion pairing agents; and combinations thereof. An amphiphilic entity component may be a mixture of different amphiphilic entities. Those skilled in the art will recognize that this is an exemplary, not comprehensive, list of substances with surfactant activity. Any amphiphilic entity may be used as a particle forming material in the production of synthetic nanocarriers to be used in accordance with the present invention.

In some embodiments, the amphiphilic polymer is one of the above mentioned polymers with a hydrophobic portion coupled to a hydrophilic pharmaceutical agent or a hydrophilic targeting moiety. In one embodiment, a polymer is coupled to a targeting moiety and another polymer is coupled to a pharmaceutical agent such as a B cell antigen.

In some embodiments, particle forming material and formed synthetic nanocarriers may optionally comprise one or more carbohydrates. Carbohydrates may be natural or synthetic. A carbohydrate may be a derivatized natural carbohydrate. In certain embodiments, a carbohydrate comprises monosaccharide or disaccharide, including but not limited to glucose, fructose, galactose, ribose, lactose, sucrose, maltose, trehalose, cellbiose, mannose, xylose, arabinose, glucoronic acid, galactoronic acid, mannuronic acid, glucosamine, galatosamine, and neuramic acid. In certain embodiments, a carbohydrate is a polysaccharide, including but not limited to pullulan, cellulose, microcrystalline cellulose, hydroxypropyl methylcellulose (HPMC), hydroxycellulose (HC), methylcellulose (MC), dextran, cyclodextran, glycogen, starch, hydroxyethylstarch, carageenan, glycon, amylose, chitosan, N,O-carboxylmethylchitosan, algin and alginic acid, starch, chitin, heparin, konjac, glucommannan, pustulan, heparin, hyaluronic acid, curdlan, and xanthan. In certain embodiments, the carbohydrate is a sugar alcohol, including but not limited to mannitol, sorbitol, xylitol, erythritol, maltitol, and lactitol. In embodiments, the inventive synthetic nanocarriers do not comprise (or specifically exclude) carbohydrates, such as a polysaccharide.

“Pharmaceutically acceptable carrier” means a pharmacologically inactive material used together with the recited synthetic nanocarriers to formulate the inventive compositions. Pharmaceutically acceptable carriers comprise a variety of materials known in the art, including but not limited to saccharides (such as glucose, lactose, and the like), preservatives such as antimicrobial agents, reconstitution aids, colorants, saline, and buffers.

“Subject” means animals, including warm blooded mammals such as humans and primates; avians; domestic household or farm animals such as cats, dogs, sheep, goats, cattle, horses and pigs; laboratory animals such as mice, rats and guinea pigs; fish; reptiles; zoo and wild animals; and the like.

“Synthetic nanocarrier(s)” means a discrete object that is not found in nature, and that possesses at least one dimension that is less than or equal to 5 microns in size. Synthetic nanocarriers according to the invention comprise one or more surfaces, including but not limited to internal surfaces (surfaces generally facing an interior portion of the synthetic nanocarrier) and external surfaces (surfaces generally facing an external environment of the synthetic nanocarrier). Synthetic nanocarriers according to the invention that have a minimum dimension of equal to or less than about 100 nm, do not comprise a surface with hydroxyl groups that activate complement or alternatively comprise a surface that consists essentially of moieties that are not hydroxyl groups that activate complement. In a preferred embodiment, synthetic nanocarriers according to the invention that have a minimum dimension of equal to or less than about 100 nm, do not comprise a surface that substantially activates complement or alternatively comprise a surface that consists essentially of moieties that do not substantially activate complement. In a more preferred embodiment, synthetic nanocarriers according to the invention that have a minimum dimension of equal to or less than about 100 nm, preferably equal to or less than about 100 nm, do not comprise a surface that activates complement or alternatively comprise a surface that consists essentially of moieties that do not activate complement. In embodiments, synthetic nanocarriers may possess an aspect ratio greater than 1:1, 1:1.2, 1:1.5, 1:2, 1:3, 1:5, 1:7, or greater than 1:10.

“Targeting feature” means any agent or molecular configuration of which the inventive synthetic nanocarriers are comprised that binds specifically to a moiety on a cell or a tissue, whereby a synthetic nanocarrier may be targeted to such a cell (or like population of cells) or tissue. Targeting features include proteins, peptides, glycoproteins, lipids, carbohydrates, receptor agonists, receptor antagonists, receptor ligands and binding pairs, antibodies, portions of antibodies, B cell antigens, and the like. The Targeting feature may be an APC targeting feature.

“Targeting moiety” means a Targeting feature separate from a synthetic nanocarrier.

“T cell antigen” means any antigen that is recognized by and triggers an immune response in a T cell (e.g., an antigen that is specifically recognized by a T cell receptor on a T cell or an NKT cell via presentation of the antigen or portion thereof bound to a Class I or Class II major histocompatability complex molecule (MHC), or bound to a CD1 complex. In some embodiments, an antigen that is a T cell antigen is also a B cell antigen. In other embodiments, the T cell antigen is not also a B cell antigen. T cells antigens generally are proteins or peptides. T cell antigens may be an antigen that stimulates a CD8+ T cell response, a CD4+ T cell response, or both. The nanocarriers, therefore, in some embodiments can effectively stimulate both types of responses. In some embodiments the T cell antigen is a ‘universal’ T cell antigen (i.e., one which can generate an enhanced response to an unrelated B cell antigen through stimulation of T cell help). In embodiments, a universal T cell antigen may comprise one or more peptides derived from tetanus toxoid, Epstein-Barr virus, influenza virus, or a Padre peptide.

### DETAILED DESCRIPTION OF INVENTION

The present invention generally relates to emulsions, including double and multiple emulsions, as well as techniques for making and using such emulsions. In one aspect, the present invention is generally directed to systems and methods for creating double and other multiple emulsions which contain more than one type of droplet. For example, the emulsion may contain a first child droplet and a second child droplet that is different from first child droplet in its composition. The first child droplet may contain a first species, and the second child droplet may contain a second species that is different in composition from the first species. In some cases, the first species and the second species can interact with or affect each other when in the same solution. For example, during one experiment, combination of a solution of oligonucleotide and a solution of a peptide resulted in immediate precipitation. To avoid this interaction, the first child droplet may be free or substantially free of the second species, and/or the second child droplet may also be free or substantially free of the first species. By keeping the first and second species in separate droplets, the effect of the first and second species respecting one another during the manufacturing process is avoided or at least minimized. By substantially free, it is meant free to an extent where the unwanted interaction of the two or more species is avoided or at least minimized. Substantially free includes a level that is undetectable or de minimus.

It will be understood that there may be additional species in additional child droplets. For example, a parent droplet may be manufactured to contain a first species in a first child droplet, a second species different from the first species in a second child droplet, a third species different from the first and second species in a third child droplet, and so on. Likewise the various child droplets may be different in relative concentrations of the same species. For example, the first child droplet may contain a first species at a relatively low concentration and a second species a relatively high concentration, whereas the second child droplet may contain the first species at a relatively high concentration and a second species at a relatively low concentration. In embodiments, the concentrations of the species in the first child droplet and the second child droplet may differ by at least about 10%, at least about 20%, at least about 30%, at least about 50%, at least about 75%, at least about 100%, at least about 200%, at least about 500%, etc., where the percentage is taken relative to the smaller of the two concentrations being compared.

Synthetic nanocarriers are then formed using the foregoing emulsions. The parent droplets are manufactured to contain particle forming material. The parent droplets (which contain the child droplets) are solidified, typically by extraction of at least a portion of the fluid of the parent droplets, such that synthetic nanocarriers are formed of the particle forming material. Without wishing to be bound by any theory of the invention, it is believed that as the synthetic nanocarriers are formed, they capture or encapsulate the species dissolved or suspended in the child droplets, thereby forming synthetic nanocarriers containing such species.

There are various ways to make the double emulsions of the invention. One method involves forming a first emulsion by combining a first aqueous liquid containing a first species with a second liquid that is immiscible with the first liquid under conditions to create the first emulsion, wherein the second liquid is the continuous phase of the first emulsion. The first species typically is dissolved in the first liquid, although the first species could be suspended or otherwise dispersed in the first liquid. A second emulsion is formed by combining a third aqueous liquid containing a second species with a fourth liquid that is immiscible with the third aqueous liquid under conditions to create the second emulsion, wherein the fourth liquid is the continuous phase of the second emulsion. The second species typically is dissolved in the third liquid, although the second species could be suspended or otherwise dispersed in the third liquid. In this method, the second and the fourth liquids contain particle forming material, and the second liquid and the fourth liquid are miscible. A third emulsion is then formed by combining the first emulsion, the second emulsion and a fifth aqueous fluid that is immiscible with the mixture of the second and the fourth liquids under conditions to create the third emulsion, wherein the fifth fluid is the continuous phase of the third emulsion. This third emulsion is a double emulsion of the invention. This emulsion has as a continuous phase the fifth aqueous liquid. This liquid contains as a dispersed phase parent droplets. The parent droplets are of the second and fourth fluids, and contain child droplets which in turn contain the first species and the second species. It is believed that the droplets of the first species contain little or no amount of the second species and that the droplets of the second species contain little or no amount of the first species. Then the second and fourth liquids are extracted to form synthetic nanocarriers containing the first and the second species.

In one embodiment, the first and the second emulsions are mixed to create a mixed emulsion which has droplets containing the first species and droplets containing the second species. Both types of droplets are in a continuous phase that is a mixture of the second and fourth liquids. This mixture then is combined with the fifth fluid to form the third emulsion, which has as a continuous phase the fifth fluid and as a dispersed phase parent droplets of the mixture of the second and fourth liquids. These parent droplets in turn have as a continuous phase the mixture of the second and the fourth liquids and have as a dispersed phase droplets of the first species and droplets of the second species. Then one or both of the second liquid and the fourth liquid are extracted to form synthetic nanocarriers containing the first species and the second species.

It will be understood that the particle forming material can be self assembling material, whereby the formed synthetic nanocarriers have particular properties. For example, the particle forming material can include amphiphilic polymers of a hydrophobic region and a hydrophilic region. The hydrophilic region can be a moiety such as a targeting moiety, B Cell antigen, or other surface active molecule. As such, the formed synthetic nanocarrier will self assemble to carry the targeting moiety, B Cell antigen, or other surface active molecule on its surface. Such self assembling particle forming material is described in PCT publication number WO 2009/051837 to von Andrian et al. Alternatively, such targeting moieties, B cell antigens, or other molecules as desired may be absorbed on or coupled to the surface of the synthetic nanocarriers after the synthetic nanocarriers are formed.

Certain aspects of the present invention are generally directed to double emulsions. As mentioned, a typical “double emulsion” comprises a plurality of discrete ‘child droplets’ of a first fluid contained within individual, discrete ‘parent droplets’ of a second fluid, which in turn are contained within a continuous phase of a third fluid. Typically, the fluids in an emulsion of the invention are liquids. The fluid may have any suitable viscosity that permits flow of the fluid.

In a double emulsion, the first fluid is typically substantially immiscible in the second fluid, and the second fluid is typically substantially immiscible in the third fluid. One or more surfactants or other emulsifiers may be present in one or more of the fluids in order to at least partially stabilize the emulsion, e.g., against coagulation or fusion of droplets within the emulsion.

It should be understood that, as used herein, terms such as “first fluid,” “second fluid,” “third fluid,” etc., are used to distinguish the various nesting levels of fluids within an emulsion, and should not be interpreted as requiring, for example, that three or more different liquids must necessarily be present. As a specific non-limiting example, a double emulsion may be formed from a first liquid comprising water (e.g., an aqueous solution), a second liquid that is substantially immiscible in water, and a third liquid comprising water (e.g., an aqueous solution), where the third liquid may be the same or different from the first liquid. The “double emulsion” character is maintained in such an emulsion because the second liquid is substantially immiscible with the first liquid, and also the second liquid is substantially immiscible with the third liquid. However, there is no requirement in such a system that the first liquid must necessarily be substantially immiscible with the third liquid. Instead, double emulsions may be created where the first liquid is substantially immiscible with the third liquid, where the first liquid is miscible with the third liquid, or even where the first liquid is identical to the third liquid.

As used herein, two fluids are substantially immiscible with each other when one cannot be solubilized in the other to a concentration of at least 10% by weight when the fluids are left undisturbed in physical contact with each other under ambient conditions (e.g., at 25° C. and 1 atm) for at least an hour. As one non-limiting example, a double emulsion may include a first liquid that is water-soluble, a second liquid that is water-insoluble (i.e., immiscible in water, sometimes termed the “oil” phase), and a third liquid that is water-soluble, e.g., a “water/oil/water” emulsion. In another example, a first liquid may be water-insoluble, a second liquid may be water-soluble, and a third liquid may be water-insoluble, e.g., an “oil/water/oil” emulsion. It should be noted that the term “oil” in the above terminology, and as used herein, merely refers to a liquid that is not miscible in water, as is known in the art. (The oil may also be referred to as being “hydrophobic” or being a hydrophobic liquid, in some embodiments, in contrast to a hydrophilic liquid which is substantially miscible in pure water.) Thus, for example, the oil may be a hydrocarbon (e.g., octane or benzene) in some embodiments, but in other embodiments, the oil may comprise other hydrophobic compounds, for example, silicone oil or methylene chloride (CH₃Cl), which are not necessarily pure hydrocarbons. Similarly, a “water-soluble” liquid (also referred to as the “water” phase) may be pure water, an aqueous solution, or another liquid, such as ethanol, that is soluble or miscible in water. Thus, the terminology “water” and “oil” should be understood as shorthand for a phase comprising a water-soluble liquid and a phase comprising a water-insoluble liquid.

One example of a set of three mutually immiscible fluids is silicone oil, a mineral oil, and an aqueous solution. Another example is a silicone oil, a fluorocarbon oil, and an aqueous solution. Yet another example is a hydrocarbon oil (e.g., hexadecane), a fluorocarbon oil, and an aqueous solution. Non-limiting examples of suitable fluorocarbon oils include octadecafluorodecahydronaphthalene or 1-(1,2,2,3,3,4,4,5,5,6,6-undecafluorocyclohexyl)ethanol.

The emulsions may be formed by applying energy in conventional ways. Many techniques for forming emulsions are known by those of ordinary skill in the art, and include, for example, by mixing or homogenizing two substantially immiscible fluids together. In some cases, high-speed mixing or sonication may be used to facilitate creation of the emulsion. Sonication typically involves applying sound (usually ultrasound) energy to a sample to facilitate mixing. Many types of suitable sonication transducers can be commercially obtained.

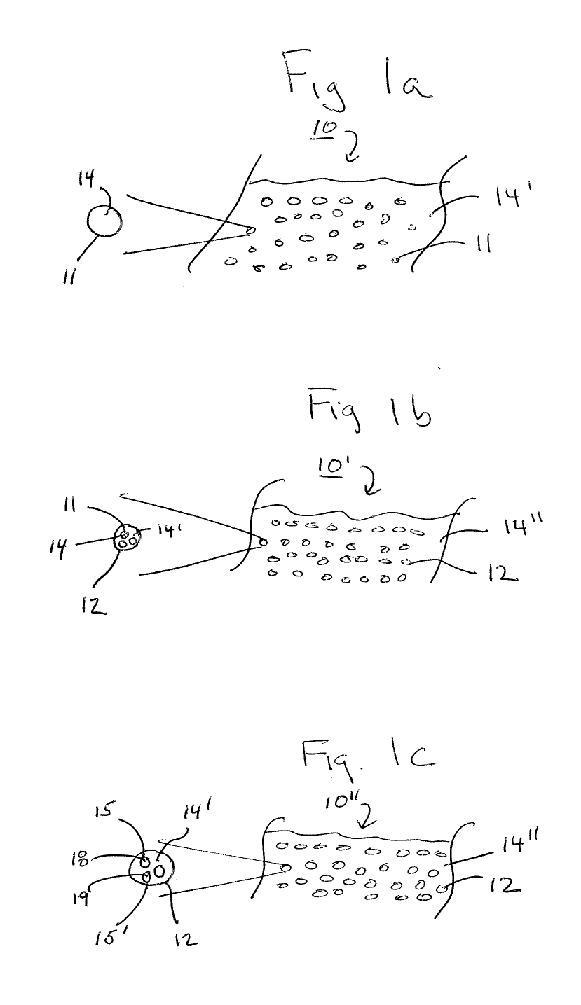

Schematic examples of emulsions are shown in FIG. 1. In FIG. 1A, a single emulsion 10 is formed from discrete droplets 11 of a first fluid 14 forming a dispersed phase of emulsion 10. The droplets 11 are contained within a second fluid 14′ forming a continuous phase of the emulsion.

In FIG. 1B, a double emulsion 10′ is formed from discrete droplets 11 of a first fluid 14, contained within parent droplets 12. The droplets 11 are dispersed in a second fluid 14′ comprising the parent droplets 12 and forming a continuous phase of the droplet 12. The parent droplets 12, in turn, are contained within a third fluid 14″ forming a continuous phase of the emulsion 10′.

According to the invention, the double emulsion comprises parent droplets which contain more than one type of child droplet, where the child droplets are distinguishable at least on the basis of the material dissolved, mixed or suspended in them. For example, in FIG. 1C, a double emulsion 10″ is depicted. First child droplets 15 contain a first species 18 dissolved in first fluid 14. Second child droplets 15′ contain a second species 19 dissolved in first fluid 14. The first fluid 14 in droplet 15 may be the same or different from the first fluid 14 in droplet 15′. The child droplets 15 & 15′ are dispersed in a second fluid 14′ comprising parent droplets 12, which in turn are dispersed within third fluid 14″ forming a continuous phase of the emulsion 10″. Techniques for forming such double emulsions containing distinguishable child droplets are discussed in more detail below.

In higher-order multiple emulsions, e.g., a triple emulsion, the nesting layer of droplets in which the layer includes first droplets and second droplets that are distinguishable may be, but need not be, the innermost (i.e., child) droplets. For example, in a triple emulsion of child droplets, parent droplets, and grandparent droplets, there may be first and second distinguishable child droplets, and/or first and second distinguishable parent droplets. Accordingly, it should be understood that, in the descriptions herein, reference to double emulsions or distinguishable child droplets are by way of example only, and in other embodiments, other multiple emulsions may be present, and/or the distinguishable droplets may be of any suitable order of droplets within the multiple emulsion, not necessarily the innermost or child droplets.

If a species is present in a droplet (in any suitable nesting level of droplets), it may be present in any suitable form, for example, dissolved, mixed, and/or suspended within the droplet. The species may be present within the droplet dissolved, as a solid or as a gas in some instances.

In some embodiments, the first species and the second species may interact with each other when in contact, for example, to precipitate out of solution at certain concentrations. By keeping the first and second species in separate droplets (for example, in separate child droplets), however, the effect of the first species on the second species is avoided or at least minimized. As a specific example, the first species may be an oligonucleotide, and the second species may be a peptide or a protein.

It should be noted that, as used herein, an “interaction” is not necessarily limited to only those interactions in which precipitation occurs. Interaction can also include chemical interactions where covalent bonds are formed or broken, ionic interactions, hydrophobic forces, van der Waals effects, or the like. Such interactions may be reversible or non-reversible, and may be equilibrium-based in some embodiments.

The undesirable interaction of a first and second species can be determined, for instance, by creating a fluid phase, such as a liquid phase, containing both the first species and the second species at a desired concentration, and determining if an undesirable interaction occurs in the fluid phase. An undesirable interaction also may occur during the emulsion forming process or synthetic nanocarrier forming process of the invention.

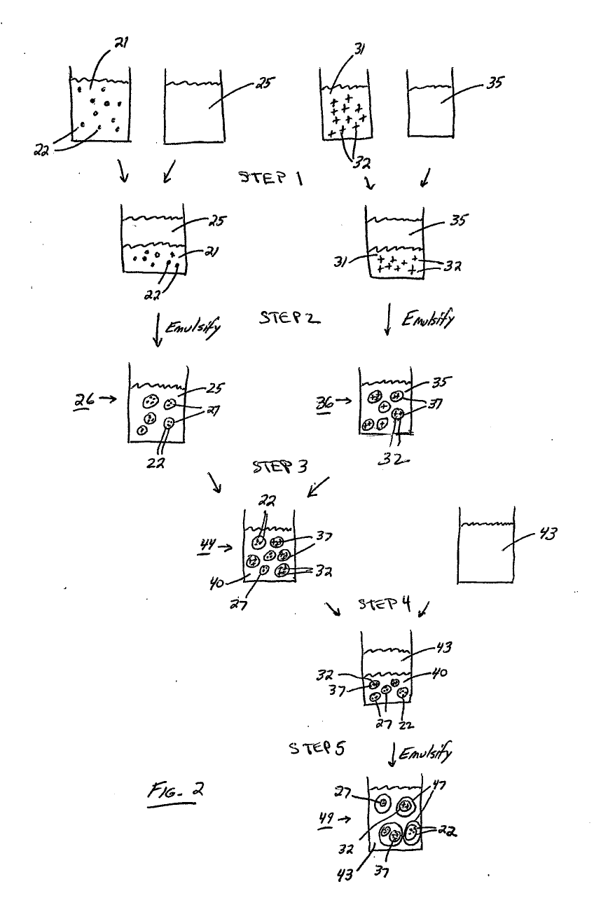

An example of a process according to the invention is illustrated schematically in FIG. 2. In step 1, a first fluid 21 containing a first species 22 is exposed to a first carrying fluid 25, which is substantially immiscible with first fluid 21. Due to their immiscibility, the fluids do not mix. In step 2, the fluids are mixed and/or emulsified (e.g., using sonication) to form a first emulsion 26, with the first fluid 21 (containing first species 22) being contained in droplets 27 within first carrying fluid 25. (In some embodiments, first fluid 21 and first carrying fluid 25 may be immediately mixed to form the emulsion, without allowing an intermediate phase-separated state to form such as is shown in the figure. In addition, if phase separation does occurs, either of the fluids may be the one on top, depending on the particular fluids used.) Step 1 and step 2 are duplicated using second fluid 31, second species 32, and second carrying fluid 35, which is substantially immiscible with second fluid 31. This creates second emulsion 36 with droplets 37 containing second fluid 31 (and species 32). As mentioned above, first carrying fluid 25 and second carrying fluid 35 may be miscible, and they may the same or different fluids. In some cases, second fluid 31 may be the same or different from first fluid 21.

As is shown in step 3, first emulsion 26 and second emulsion 36 may then be mixed together in some fashion to create combined emulsion 44 comprising first droplets 27 containing first species 22 and second droplets 37 containing second species 32 in continuous fluid 40, where continuous fluid 40 is formed by mixing the first carrying fluid and the second carrying fluid together (e.g., where the first carrying fluid and the second carrying fluid are miscible with each other). Emulsion 44 may be prepared using any suitable technique, e.g., by simple pouring of one emulsion into another, optionally with other techniques such as mixing, homogenization, sonication, etc., for example, as described herein.

In step 4, emulsion 44 may be exposed to a third fluid 43 that is substantially immiscible with emulsion 44. This can be seen schematically in FIG. 2 with a phase-separated system of continuous fluid 40 and third fluid 43. (As above, these may be immediately mixed to form an emulsion, without allowing an intermediate phase-separated state such as is shown in FIG. 2 to form.) Third fluid 43 may or may not be substantially miscible with first fluid 21 and/or second fluid 31, present in discrete droplets contained within continuous fluid 40. The third fluid and the emulsion may be then be mixed and/or emulsified (e.g., using sonication) to form a double emulsion 49, with discrete, first and second child droplets 27 and 37, containing respective first fluid 21 and second fluid 31 (and containing respectively first species 22 and second species 32), where the child droplets are contained within parent droplets 47 of the third fluid, which in turn are contained within a continuous third fluid 43. Each parent droplet 47 may contain one or more child droplets, and each parent droplet 47 may contain one or both types of child droplets (27 and 37).

In one aspect, synthetic nanocarriers may be formed using emulsions such as those discussed herein, including double emulsions or other multiple emulsions. For example, in a double emulsion comprising child droplets of a first fluid contained within parent droplets of a second fluid, contained within a continuous third fluid, at least some of the fluid from the parent droplets may be removed or extracted, causing the parent droplets to decrease in size. For example, extraction of liquid from parent droplets of a double emulsion may occur due to liquid/liquid or liquid/air extraction processes, such as those known to ordinary skill in the art, including dilution, evaporation, reduced-pressure evaporation, spray drying, lyophilization, or supercritical fluid extraction. In some cases, enough liquid may be extracted such that synthetic nanocarriers, are formed from the parent droplets.

As a specific non-limiting example, a double emulsion may be prepared where the child droplets comprise an aqueous solution, the parent droplets comprise a polymer in methylene chloride, and the continuous liquid comprises an aqueous solution. Methylene chloride is substantially immiscible in water; however, over time, at least some of the methylene chloride can be extracted into the continuous liquid, thereby causing at least some of the parent droplets to shrink, and form synthetic nanocarriers in some cases. Examples of suitable polymers include, but are not limited to, polylactic acid, polyglycolic acid, poly(lactic-co-glycolic acid), polyethylene glycol, polyanhydrides, polyorthoesters, polyurethanes, polybutyric acid, polyvaleric acid, polylactide-co-caprolactone, polycarbonate, polymethacrylic acid, polyethylenevinyl acetate, polytetrafluorethylene, polymethyl methacrylate, polyacrylic acid, polyesters, or the like. Other examples of suitable solvents for the parent droplets include, but are not limited to, chloromethane or methylene bromide.

Synthetic nanocarriers formed as described above may have any suitable diameter, for example, between about 100 and 1500 nanometers. Particular sizes are discussed above and examples are described in the Example section below. Synthetic nanocarrier sizes may be determined using any suitable technique, for example, using a Brookhaven ZetaPALS. Using a Brookhaven ZetaPALs, size is referred to as an average diameter which is weighted by the intensity of light scattered by each nanocarrier. Once formed, the synthetic nanocarriers can be removed or separated from the containing fluids in some embodiments. Any suitable separation technique may be used to separate the synthetic nanocarriers. For example, the synthetic nanocarriers can be removed via sedimentation, filtration, extraction, evaporation (e.g., of a liquid containing the synthetic nanocarriers), or the like.

Without wishing to be bound by any theory, it is believed that such synthetic nanocarriers, once formed, comprise first regions and second regions that are distinguishable in some fashion, e.g., compositionally as previously described. For example, the synthetic nanocarrier may include first regions that are defined, at least in part, by the first species, and second regions that are defined, at least in part, by the second species. In some embodiments, the first regions are substantially free of the second species and/or the second regions are substantially free of the first species. It is believed that such synthetic nanocarriers are able to form due to a lack of complete mixing that occurs between the first and second species as the parent droplets shrink to form synthetic nanocarriers.

In one aspect, the present invention also provides any of the above-mentioned compositions in kits, optionally including instructions for use of the composition. “Instructions” typically involve written instructions on or associated with packaging of compositions of the invention. Instructions also can include any oral or electronic instructions provided in any manner. The “kit” typically defines a package including any one or a combination of the compositions of the invention and optionally the instructions. The kits described herein may also contain one or more containers, which may contain the inventive composition and other ingredients as previously described. The kits also may contain instructions for mixing, diluting, and/or administrating the compositions of the invention in some cases. The compositions of the kit may be provided as any suitable form, for example, as liquid solutions or as dried powders or synthetic nanocarriers.

While several embodiments of the present invention have been described and illustrated herein, those of ordinary skill in the art will readily envision a variety of other means and/or structures for performing the functions and/or obtaining the results and/or one or more of the advantages described herein, and each of such variations and/or modifications is deemed to be within the scope of the present invention. More generally, those skilled in the art will readily appreciate that all parameters, dimensions, materials, and configurations described herein are meant to be exemplary and that the actual parameters, dimensions, materials, and/or configurations will depend upon the specific application or applications for which the teachings of the present invention is/are used. Those skilled in the art will recognize, or be able to ascertain using no more than routine experimentation, many equivalents to the specific embodiments of the invention described herein. It is, therefore, to be understood that the foregoing embodiments are presented by way of example only and that, within the scope of the appended claims and equivalents thereto, the invention may be practiced otherwise than as specifically described and claimed. The present invention is directed to each individual feature, system, article, material, kit, and/or method described herein. In addition, any combination of two or more such features, systems, articles, materials, kits, and/or methods, if such features, systems, articles, materials, kits, and/or methods are not mutually inconsistent, is included within the scope of the present invention.

The indefinite articles “a” and “an,” as used herein in the specification and in the claims, unless clearly indicated to the contrary, should be understood to mean “at least one.” For example, reference to “a polymer” includes a mixture of two or more such molecules, reference to “a solvent” includes a mixture of two or more such solvents, reference to “an adhesive” includes mixtures of two or more such materials, and the like.

The phrase “and/or,” as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified. Thus, as a non-limiting example, a reference to “A and/or B”, when used in conjunction with open-ended language such as “comprising” can refer, in one embodiment, to A only (optionally including elements other than B); in another embodiment, to B only (optionally including elements other than A); in yet another embodiment, to both A and B (optionally including other elements); etc.

As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (i.e. “one or the other but not both”) when preceded by terms of exclusivity, such as “either,” “one of,” “only one of,” or “exactly one of.” “Consisting essentially of,” when used in the claims, shall have its ordinary meaning as used in the field of patent law.

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

It should also be understood that, unless clearly indicated to the contrary, in any methods claimed herein that include more than one step or act, the order of the steps or acts of the method is not necessarily limited to the order in which the steps or acts of the method are recited.

In the claims, as well as in the specification above, all transitional phrases such as “comprising,” “including,” “carrying,” “having,” “containing,” “involving,” “holding,” “composed of,” and the like are to be understood to be open-ended, i.e., to mean including but not limited to. Only the transitional phrases “consisting of” and “consisting essentially of” shall be closed or semi-closed transitional phrases, respectively, as set forth in the United States Patent Office Manual of Patent Examining Procedures, Section 2111.03.

### EXAMPLES

Example 1 describes synthetic nanocarriers prepared using a standard double emulsion process, with an oligonucleotide and a peptide in the same inner aqueous phase. The solution of oligonucleotide and peptide were prepared at the highest concentration achievable without precipitation occurring due to the interaction of the oligonucleotide and the peptide. The formed nanocarriers contained detectable oligonucleotide but did not contain detectable peptide as shown in Table 1.

Example 2 describes synthetic nanocarriers prepared using a double emulsion of the invention, with an oligonucleotide and a peptide in different inner aqueous phases. The formed nanocarriers contained detectable oligonucleotide and detectable peptide as shown in Table 1.

Example 3 describes synthetic nanocarriers prepared using a double emulsion of the invention, with an oligonucleotide and a peptide in different inner aqueous phases. The formed nanocarriers contained detectable oligonucleotide and detectable peptide as shown in Table 1. This examples shows that amount of oligonucleotide encapsulated can be increased while continuing to encapsulate peptide.

TABLE 1

| | Number | | | Effective |
|---|---|---|---|---|
| | Primary | Oligonucleotide | Ovalbumin | Diameter |
| Example | Emulsions | Load (% of NC) | Load (% of NC) | (nm) |
| 1 | 1 | 0.6 | 0.0 | 222 |
| 2 | 2 | 0.6 | 0.8 | 251 |
| 3 | 2 | 3.7 | 0.3 | 204 |

### Example 1

### Standard Double Emulsion with Single Primary Emulsion

Ovalbumin peptide 323-339, a 17 amino acid peptide known to be a T and B cell epitope of Ovalbumin protein, was purchased from Bachem Americas Inc. (3132 Kashiwa Street, Torrance Calif. 90505. Part #4065609.)

A 25mer DNA oligonucleotide with a sodium counter-ion on a phosphorothioate backbone was purchased from Aveica Biotechnology (155 Fortune Boulevard, Milford, Mass. 01757. Product Code AAB.)

PLA with an inherent viscosity of 0.14 dL/g was purchased from SurModics Pharmaceuticals (756 Tom Martin Drive, Birmingham, Ala. 35211. Product Code 100 DL 1.5A.)

PLA-PEG-nicotine with a molecular weight of approximately 22,000 Da was synthesized by Selecta. See PCT publication WO 2009/051837, FIG. 30.

Polyvinyl alcohol (Mw=9,000-10,000, 80% hydrolyzed) was purchased from Sigma (Part Number 360627).

The above materials were used to prepare the following solutions:

Solution 1: Ovalbumin peptide 323-339 @ 15 mg/mL and oligonucleotide @ 100 mg/ml in dilute hydrochloric acid aqueous solution. The solution was prepared by first preparing two separate solutions at room temperature: ovalbumin peptide @ 30 mg/mL in 0.06N hydrochloric acid solution and oligonucleotide @ 200 mg/mL in 0.13N hydrochloric acid solution. An equal part of ovalbumin peptide solution was then added to the oligonucleotide solution to prepare the final solution.

Solution 2: PLA @ 50 mg/mL and PLA-PEG-nicotine @ 50 mg/ml in pure methylene chloride. The solution was prepared by first preparing two separate solutions at room temperature: PLA @ 100 mg/mL in pure methylene chloride and PLA-PEG-nicotine @ 100 mg/mL in pure methylene chloride. Equal parts of each solution were combined to prepare the final solution.

Solution 3: Polyvinyl alcohol @ 50 mg/mL in 100 mM pH 8 phosphate buffer.

The water in oil (W/O) primary emulsion was prepared by combining solution 1 (0.1 mL) and solution 2 (1.0 mL) in a small pressure tube and sonicating at 50% amplitude for 40 seconds using a Branson Digital Sonifier 250. The water/oil/water (W/O/W) double emulsion was prepared by adding solution 3 (2.0 mL) to the primary emulsion and sonicating at 15% amplitude for 15 seconds using the Branson Digital Sonifier 250.

The double emulsion was added to a beaker containing phosphate buffer solution (30 mL) and stirred at room temperature for 2 hours to allow for the methylene chloride to evaporate and for the nanocarriers to form. A portion of the nanocarriers were washed by transferring the nanocarrier suspension to a centrifuge tube and spinning at 13,823 g for one hour, removing the supernatant, and re-suspending the pellet in phosphate buffered saline. The washing procedure was repeated and the pellet was re-suspended in phosphate buffered saline for a final nanocarrier dispersion of about 10 mg/mL.

The amounts of oligonucleotide and peptide in the nanocarrier were determined.

### Example 2

### Double Emulsion with Multiple Primary Emulsions

Materials were obtained as described above in Example 1.

Solution 1: Ovalbumin peptide 323-339 @ 70 mg/mL in dilute hydrochloric acid aqueous solution. The solution was prepared by dissolving ovalbumin peptide in 0.13N hydrochloric acid solution at room temperature.

Solution 2: PLA @ 50 mg/mL and PLA-PEG-nicotine @ 50 mg/ml in methylene chloride. The solution was prepared by first preparing two separate solutions at room temperature: PLA @ 100 mg/mL in pure methylene chloride and PLA-PEG-nicotine @ 100 mg/mL in pure methylene chloride. Equal parts of each solution were combined to prepare the final solution.

Solution 3: Oligonucleotide @ 200 mg/ml in dilute hydrochloric acid aqueous solution. The solution was prepared by dissolving oligonucleotide in 0.13N hydrochloric acid solution at room temperature.

Solution 4: Same as Solution #2.

Solution 5: Polyvinyl alcohol @ 50 mg/mL in 100 mM pH 8 phosphate buffer.

Two separate primary water in oil emulsions were prepared. W1/02 was prepared by combining solution 1 (0.1 mL) and solution 2 (1.0 mL) in a small pressure tube and sonicating at 50% amplitude for 40 seconds using a Branson Digital Sonifier 250. W3/O4 was prepared by combining solution 2 (0.1 mL) and solution 4 (1.0 mL) in a small pressure tube and sonicating at 50% amplitude for 40 seconds using a Branson Digital Sonifier 250. A third emulsion with two inner emulsion ([W1/O2,W3/O4]/W5) emulsion was prepared by combining 0.5 ml of each primary emulsion (W1/O2 and W3/O4) and solution 5 (2.0 mL) and sonicating at 15% amplitude for 15 seconds using the Branson Digital Sonifier 250.

The third emulsion was added to a beaker containing phosphate buffer solution (30 mL) and stirred at room temperature for 2 hours to allow for the methylene chloride to evaporate and for the nanocarriers to form. A portion of the nanocarriers were washed by transferring the nanocarrier suspension to a centrifuge tube and spinning at 13,823 g for one hour, removing the supernatant, and re-suspending the pellet in phosphate buffered saline. The washing procedure was repeated and the pellet was re-suspended in phosphate buffered saline for a final nanocarrier dispersion of about 10 mg/mL.

The amounts of oligonucleotide and peptide in the nanocarrier were determined.

### Example 3

### Double Emulsion with Multiple Primary Emulsions

Materials were obtained as described above in Example 1, with the following exceptions.

The polyvinyl alcohol (Mw=11,000-31,000, 87-89% hydrolyzed) was purchased from Baker (Part Number U232-08).

PLA with an inherent viscosity of 0.19 dL/g was purchased from Boehringer Ingelheim Chemicals, Inc. (Petersburg, Va. Product Code R202H.)

Solution 1: Ovalbumin peptide 323-339 @ 70 mg/mL in dilute hydrochloric acid aqueous solution. The solution was prepared by dissolving ovalbumin peptide in 0.13N hydrochloric acid solution at room temperature.

Solution 2: PLA @ 75 mg/mL and PLA-PEG-nicotine @ 25 mg/ml in methylene chloride. The solution was prepared by first preparing two separate solutions at room temperature: PLA @ 100 mg/mL in pure methylene chloride and PLA-PEG-nicotine @ 100 mg/mL in pure methylene chloride. The final solution was prepared by adding 3 parts PLA solution for each part of PLA-PEG-nicotine solution.

Solution 3: Oligonucleotide @ 200 mg/ml in purified water. The solution was prepared by dissolving oligonucleotide in purified water at room temperature.

Solution 4: Same as Solution #2.

Solution 5: Polyvinyl alcohol @ 50 mg/mL in 100 mM pH 8 phosphate buffer.

Two separate primary water in oil emulsions were prepared. W1/O2 was prepared by combining solution 1 (0.1 mL) and solution 2 (1.0 mL) in a small pressure tube and sonicating at 50% amplitude for 40 seconds using a Branson Digital Sonifier 250. W3/O4 was prepared by combining solution 2 (0.1 mL) and solution 4 (1.0 mL) in a small pressure tube and sonicating at 50% amplitude for 40 seconds using a Branson Digital Sonifier 250. A third emulsion with two inner emulsion ([W1/O2,W3/O4]/W5) emulsion was prepared by combining 0.5 ml of each primary emulsion (W1/O2 and W3/O4) and solution 5 (2.0 mL) and sonicating at 30% amplitude for 40 seconds using the Branson Digital Sonifier 250.

The third emulsion was added to a beaker containing phosphate buffer solution (30 mL) and stirred at room temperature for 2 hours to allow for the methylene chloride to evaporate and for the nanocarriers to form. A portion of the nanocarriers were washed by transferring the nanocarrier suspension to a centrifuge tube and spinning at 13,823 g for one hour, removing the supernatant, and re-suspending the pellet in phosphate buffered saline. The washing procedure was repeated and the pellet was re-suspended in phosphate buffered saline for a final nanocarrier dispersion of about 10 mg/mL.

The amounts of oligonucleotide and peptide in the nanocarrier were determined.

What is claimed is:

1. A method comprising,
combining a first fluid containing a first species with a second fluid that is immiscible with the first fluid under conditions to create a first emulsion, wherein the second fluid is the continuous phase of the first emulsion,
combining a third fluid containing a second species with a fourth fluid that is immiscible with the third fluid under conditions to create a second emulsion, wherein the fourth fluid is the continuous phase of the second emulsion,
wherein the second fluid and the fourth fluid contain particle forming material,
wherein the second fluid and the fourth fluid are miscible,
combining the first emulsion, the second emulsion and a fifth fluid that is immiscible with the second fluid and the fourth fluid under conditions to create a third emulsion, wherein the fifth fluid is the continuous phase of the third emulsion, and
extracting at least a portion of one or both of the second fluid and the fourth fluid to form synthetic nanocarriers containing the first species and the second species.

2. The method of claim 1, wherein the first fluid, just prior to contact with the second fluid to form the first emulsion, is substantially free of the second species and wherein the third fluid, just prior to contact with the fourth fluid to form the second emulsion, is substantially free of the first species.

3. The method of claim 2, wherein the first species and the second species, at their concentration in the first and third fluids, interact in a solution of either one, both or a mixture of the first and third fluids.

4. The method of claim 1, wherein the third emulsion is formed by combining the first and second emulsions to form a mixture and then combining the mixture with the fifth fluid to form the third emulsion.

5. The method of claim 1, wherein the first fluid and the third fluid are miscible.

6. The method of claim 1, wherein the first, third and fifth fluids are aqueous.

7. The method of claim 1, wherein the first species is an oligonucleotide.

8. The method of claim 1, wherein the second species is a peptide.

9. The method of claim 1, wherein the particle forming material is a polymer.

10. The method of claim 1, wherein the particle forming material is a biodegradable polymer.

11. The method of claim 1, wherein the particle forming material comprises a biodegradable polymer with a hydrophobic portion and a hydrophilic portion.

12. The method of claim 1, wherein the first species is dissolved in the first fluid.

13. The method of claim 1, wherein the first species is dispersed in the first fluid.

14. The method of claim 1, wherein the synthetic nanocarriers formed have an effective diameter of between 100 and 1500 nanometers using a Brookhaven ZetaPALS.

15. The method of claim 1, wherein the synthetic nanocarriers formed have an effective diameter of between 150 and 500 nanometers using a Brookhaven ZetaPALS.

16. The method of claim 1, wherein the first species is an adjuvant.

17. The method of claim 1, wherein the second species is a T cell antigen.

18. The method of claim 1, wherein the first species is an adjuvant and the second species is a T cell antigen.

19. The method of claim 18, wherein the particle forming material comprises a biodegradable polymer with a hydrophobic portion and a hydrophilic portion.

20. The method of claim 19, wherein the first fluid is an aqueous solution of the first species and the third fluid is an aqueous solution of the second species.

21. The method of claim 20, wherein the particle forming material comprises PLA or PLGA.

22. A product prepared by the process of claim 1.
